(19) United States
(12) Patent Application Publication
(10) Pub. No.: US 2007/0244491 A1
(43) Pub. Date: Oct. 18, 2007

(54) SKIN CONDITION REMOVER WITH HANDLE

(76) Inventor: JENIFER M. RUSSELL, Maplewood, MO (US)

Correspondence Address: CHARLES C. MCCLOSKEY, 763 S. NEW BALLAS ROAD STE. 170, ST. LOUIS, MO, 63141, US

(21) Appl. No.: 11/379,102

(22) Filed: Apr. 18, 2006

(51) Int. Cl. A61B 17/50

(52) U.S. Cl. 606/131

(57) ABSTRACT

The skin conditioner remover provides a tool for a stylist to use upon a person's foot. The remover cuts off the rough skin, or callus, that may form on the big toe or elsewhere on the foot. The skin conditioner remover has two curved razor blades joined to a handle with an anti-slip surface and a thumb grip. The slightly curved head better matches the contours of a person's foot. The remover has a disposable version with a fixed head. And it has a durable version where the head detaches from the handle for replacement. About three inches long, the remover can be used at home or in a salon.

### BACKGROUND OF THE INVENTION

The present invention relates to personal skin hygiene devices in general and more particularly to a callus remover. This remover has two parallel and curved razor blades in a head upon a handle. The head and blades cooperate to remove dead skin scraped from the vicinity of a callus.

From time to time, shoes do not fit well and parts of a foot regularly contact a tougher surface. In those situations, a foot develops a callus as a protective measure. The callus is a thickening of skin tissue at the surface of the foot, generally made of dead cells. A callus continues to grow as poorly fit shoes or tougher surfaces wear upon the callus. However, a callus may appear unsightly when a person wears open shoes or may cause foot problems if it grows too much.

People endure calluses or take care of them. People take care of calluses using over the counter remedies and the occasional nail clipper or other cutting tool. For tougher calluses, people, often women, seek removal of calluses at styling salons. For more advanced cases, people seek medical attention for calluses. The present invention provides a device for salon or home use that readily removes calluses.

At the salon, patrons present their feet with calluses for removal, often as part of a pedicure. The stylist positions a foot for access to a callus. Then using a razor, the stylist removes the callus in multiple swaths while exercising caution to avoid injuring live adjacent skin. Cuttings from the callus fall to the floor below the foot for collection in a towel or other container. When finished removing a callus, the stylist returns the razor to a drawer for future use and disposes of the cuttings. This common practice raises doubts among salon patrons about sanitation of the razor in callus removal.

### DESCRIPTION OF THE PRIOR ART

Over time, various callus removers have sought to trim conditions on feet or to soften feet in a sanitary manner. The prior art removers are generally for a surgeon's use upon a patient's body or a person's use upon her own feet.

The patent to Gilhuas, U.S. Pat. No. 4,537,207 describes a callus removing skin file. The file has a narrow handle with a rasping foil attached to an end of the handle. The rasping foil has upturned longitudinal edges that engage slots in the head of the handle. The foil allows a user to grind away a callus or other rough patch of skin as the foil is moved lengthwise. However, the present invention has two curved blades for cutting a callus though the blades are oriented transverse to the direction of movement of the blades.

The patent to Segal, U.S. Pat. No. 5,624,451 shows a flexible blade that removes skin lesions and other conditions. This blade has a thin planar member that deflects when grasped by a surgeon. The blade has two parallel sharpened edges within the lobes used for finger grips. Unlike the present invention, this blade has no handle and applies one sharpened edge to a lesion where the present invention applies two edges.

Similar to Segal, Tipton's patent, U.S. Pat. No. 5,555,892 has a biopsy shaver with a thin flexible blade but having parallel plastic handles upon the lateral edges. The blade has a sharpened leading edge and an opposite ruler for estimating calluses and other skin lesions. The handles here have an upward bend that directs the blade to bend downward when the patented device is grasped. This patent differs from the present invention as it lacks a handle for grasping in the palm of the user's hand and only has a single cutting edge.

Again similar to Tipton but older, the U.S. Pat. to Hartlaub et al., U.S. Pat. No. 4,943,295 has a thin blade surgical cutting tool with finger grips. This patent has finger grips shown upon the lateral edges of a thin blade. The grips exceed the thickness of the blade and allow a surgeon to flex the blade, curved upwards or downwards. The tool also includes a guard that can bend along with the blade.

This is not the present invention of a handle with a two curved blade head.

Then the U.S. Pat. to Mallory, U.S. Pat. No. 1,477,274, shows a corn eradicator.

The eradicator has a generally rectangular shape with a blunt nose extending longitudinally. Two handles unfold upwards and extend parallel for gripping when dragging the eradicator across a corn. A spacer spans between the handles at the eradicator for stiffening during usage. Following usage, the spacer is folded down, and the handles are folded down and outwards thus making the eradicator compact. The eradicator though lacks blades as in the present invention.

Lastly, the U.S. Pat. to McCool et al., No. D392,738 shows a flexible surgical razor. This razor has a generally I shape with a blade upon one side of the web of the I shape. The flanges of the I shape are upturned with alternating tab like grips for assisting the grasp of a surgeon. This is not the present invention where two curved blades in a head attach to a handle similar to a T shape.

The present invention overcomes the limitations of the prior art explained above. That is, the art of the present invention allows a hand grip upon a handle connecting to a two bladed head. The blades form two parallel razor edges to remove quickly and cleanly layers of skin forming a callus or other condition.

### SUMMARY OF THE INVENTION

The skin conditioner remover provides personal care for the feet. The remover takes off the rough skin, or callus, that generally forms on the outside of a person's big toe, the ball of the foot, and the heel. The remover can be used at home or in a salon. The skin conditioner remover has either one or two razor blades joined to a handle with an anti-slip surface and a thumb grip. The remover has a generally T shape with a slightly curved head to better match the contours of a person's foot. The remover has a disposable version where the head is fixed to the handle. And it has a durable version where the head detaches from the handle for replacement by another head. The tool is approximately three inches in length and two inches in width.

There has thus been outlined, rather broadly, the more important features of the invention in order that the detailed description thereof that follows may be better understood and that the present contribution to the art may be better appreciated. Additional features of the invention will be described hereinafter and which will form the subject matter of the claims attached.

Numerous objects, features and advantages of the present invention will be readily apparent to those of ordinary skill in the art upon a reading of the following detailed description of the presently preferred, but nonetheless illustrative, embodiment of the present invention when taken in conjunction with the accompanying drawings. Before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, the phraseology and terminology employed herein are for the purpose of description and should not be regarded as limiting.

One object of the present invention is to provide a new and improved skin condition remover.

Another object is to provide such a skin condition remover that is of disposable construction to minimize the spread of disease between successive pedicure patrons.

A further object is to provide such a skin condition remover that fits comfortably into the hand of a salon stylist for precise handling when removing calluses and other skin conditions.

A further object is to provide such a skin condition remover that has an angled head so a salon stylist keeps her elbow close to her body for drawing of the invention in a downward motion.

A still further object is to provide such a skin condition remover that resists water and other salon chemicals.

A still further object of the present invention is to provide a skin conditioner remover that may be easily and efficiently manufactured and marketed.

A still further object of the present invention is to provide a skin conditioner remover that lowers the risk of laceration when changing razor blades.

A still further object of the present invention is to provide prompt removal of cuttings thus keeping the invention clear during usage.

A still further object of the present invention is to accommodate removing skin conditions upon the curved outside of a patron's big toe when drawing the invention from top to bottom, upon the curved ball and sole a patron's foot when drawing the invention lengthwise, and upon the heel of a patron's foot as the invention is drawn around the somewhat spherical heel shape.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In referring to the drawings,

FIG. 1 shows an isometric view of the skin condition remover in accordance with the present invention;

FIG. 1A shows a detailed view of the head of the present invention, particularly the blades;

FIG. 2 describes a side view of the present invention;

FIG. 3 shows an isometric view of an alternate embodiment of the skin condition remover;

FIG. 4 describes a side view of the alternate embodiment;

FIG. 5 shows a top view of the alternate embodiment showing the curved blades;

FIG. 6 shows an isometric view of an alternate embodiment of the present invention with an articulated head; and,

FIG. 7 shows a side view of the articulating head embodiment.

The same reference numerals refer to the same parts throughout the various figures.

### DESCRIPTION OF THE PREFERRED EMBODIMENT

In the preferred embodiment, the skin condition remover 1 has a handle 2 defining the longitudinal axis of the invention. The handle has an ergonomic shape for grasping in the palm of the hand. The ergonomic shape increases the comfort of the handle and lowers the risk of slipping during usage of the invention. The handle has a tip 2b and an opposite end 2a. The end is free and located towards the wrist of the user during usage. The tip joins to the head 3 that engages the skin during usage of the invention. The head has a generally rectangular shape. The head descends from the tip and has an arcuate, generally centered blade 4 fixed to the head. The blade is perpendicular to the handle and has a cutting edge 4a located away from the handle but towards the user. The cutting edge removes a callus or other skin condition when the present invention is drawn towards the user. Where the head joins the tip, the head has at least one guard 3a extending away from the handle and the head. The guard serves as a safety for the exposed cutting edge. Here opposite the tip, the head has a second guard, generally of the same height as the first guard. In the preferred embodiment, the guards are divided to pass cuttings and rounded to glide over skin. The grooves 3b forming between the divided guards extend from the perimeter of the blade to the outer edges of the head. In an alternate embodiment, the head has a slot located behind the cutting edge 4a. The slot opens towards the handle and has a width in the range of 0.01 inches to 0.10 inches. Cuttings pass through the slot and exit the head opposite the direction of drawing the remover.

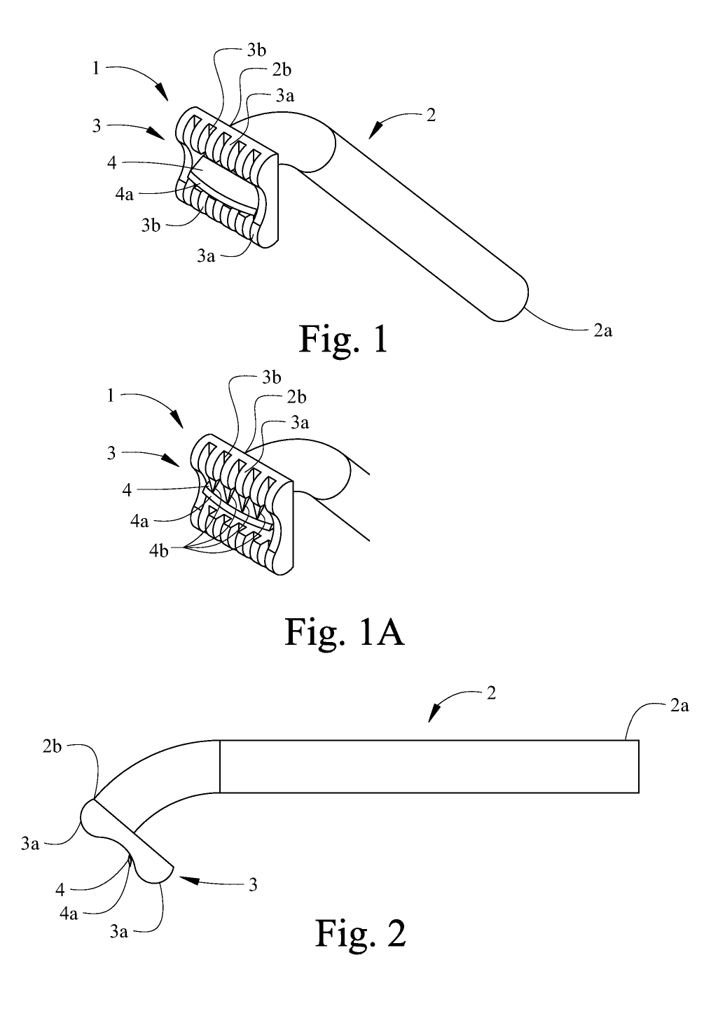

An alternate embodiment of the blade 4 on the head 3 is shown in FIG. 1A. As before, the head depends from an end 2b of the handle 2. The head has a generally rectangular shape with separated guards 3a defining groves 3b ahead and behind the blade 4. The guards and grooves are oriented in the direction of pulling the head, generally laterally upon the head. The blade remains oriented longitudinally upon the head with the cutting edge 4a perpendicular to the direction of pulling the head. The alternate embodiment has a plurality of teeth 4b, mutually parallel and spaced apart upon the surface of the blade away from the handle. Each tooth aligns with a forward and rearward guard, thus forming a continuous groove from the front to the back of the head. In usage, the head is directed towards a skin condition so that the skin condition enters a groove upon the front of the head. The skin condition passes through the forward groove and the cutting edge then removes the skin condition as a cutting. The cutting then passes between two adjacent teeth 4b and into a rearward groove. As the head proceeds forward, the cutting leaves the rearward groove 3b and falls off of the head. The salon stylist then sweeps up the cuttings as if normally cleaning a booth.

FIG. 2 shows the operation of the guards 3a upon the head 3 in a side view of the invention. The guards extend away from the head more than the cutting edges 4a. During usage, a user draws the guard, furthest from the handle, over a skin condition. In drawing the invention, the elbow of the user remains tucked into the user's body thus generating a straight stroke of the user's arm for a straight cut using the invention. The guard deflects the skin downward and then the skin rebounds to be cut by the cutting edges 4a in succession. The guard, nearest the handle, then presses the skin down to close any surface abrasions. In this figure, the head 3 attains an acute angle below the handle 2. The angle of the head orients the cutting edges towards the handle and the handle in the direction of drawing. The angle also keeps the hand of the user above the skin of the patron.

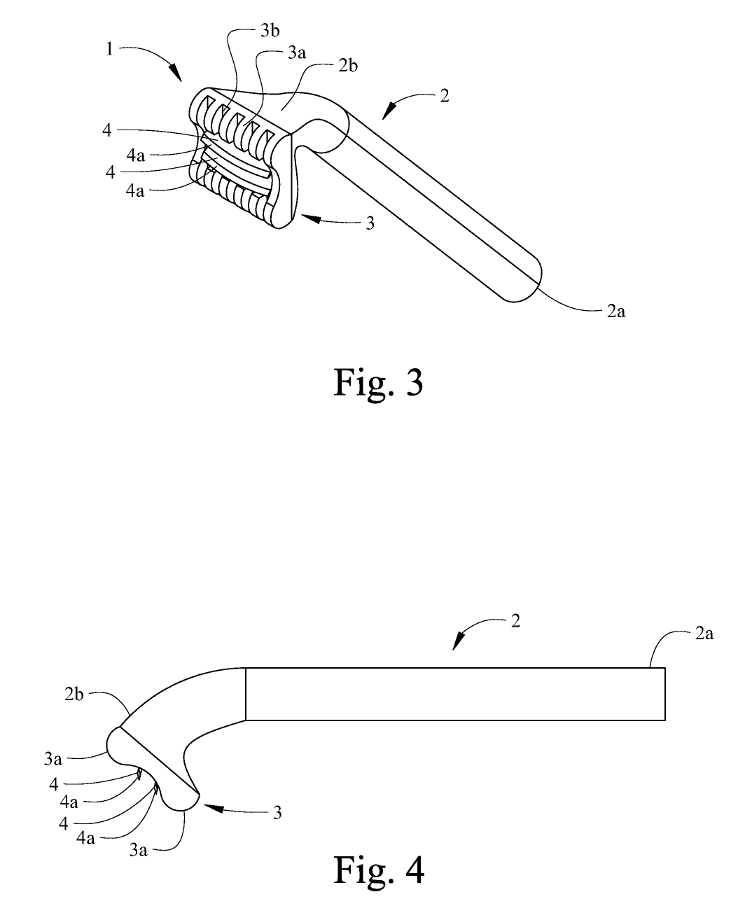

In an alternate embodiment shown in FIG. 3, the skin condition remover 1 has an ergonomic handle 2 as before. The handle has a tip 2a and an opposite free end 2b generally towards the user during usage. The tip joins to the head 3 that engages the skin during usage of the invention. The head has a generally rectangular shape, here shown as square though an oblong shape is possible. The head descends from the tip and has two parallel, spaced apart, and generally centered blades 4. The blades are arcuate and fixed to the head. The blades are perpendicular to the handle and have cutting edges 4a located away from the handle from removing a skin condition during usage of the present invention. Where the head joins the tip, the head has at least one guard 3a extending away from the handle and the head. The guard serves as a safety for the exposed cutting edges. Here opposite the tip, the head has a second guard, generally of the same height as the first guard. The guards are divided, thus forming grooves 3b to pass cuttings and the rounded shape of the guards allows the head to glide over skin.

FIG. 4 shows the operation of the guards 3a upon the head 3 in a side view of the invention. The guards extend away from the head more than the cutting edges 4a. During usage, a user draws the guard, furthest from the handle, over a skin condition which deflects and then rebounds to be cut by successive cutting edges 4a. The guard, nearest the handle, then presses the skin down to close any surface abrasions. In this embodiment, the head 3 attains an acute angle below the handle 2. The angle of the head orients the cutting edges in the direction of drawing and keeps the user's hand above the skin of the patron.

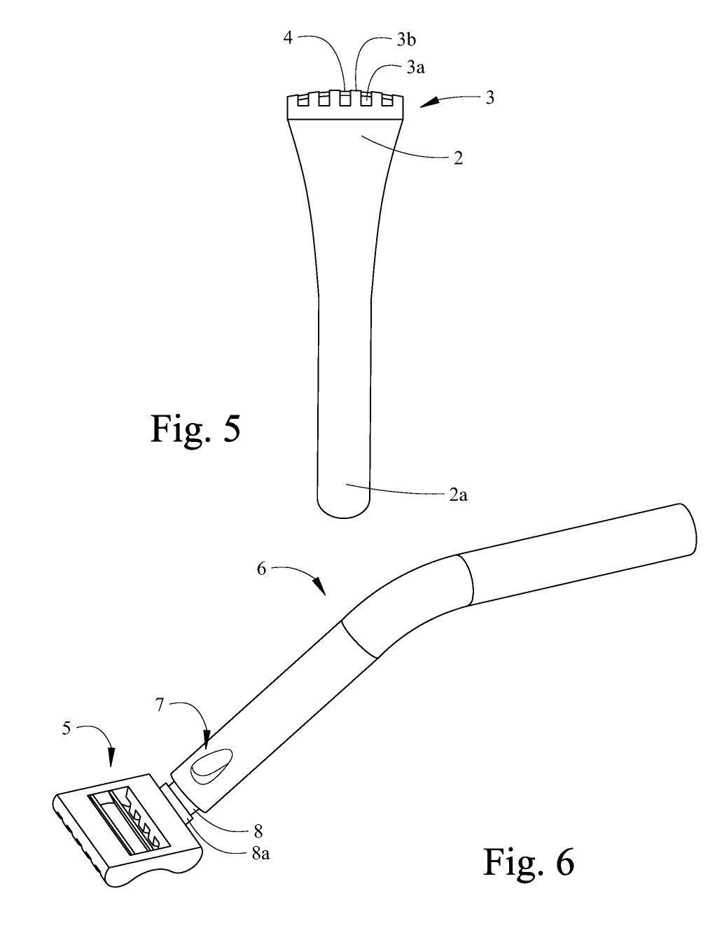

Viewing the alternate embodiment from above, the handle 2 has a taper in FIG. 5. The handle has the free end 2a away from the blade and rounded for user comfort. The handle then narrows towards the center for gripping by the user and then widens at the tip 2b. The head 3 joins to the width of the tip 2b. In the preferred embodiment, the head is molded, or formed, as a single piece with the handle. The head has a generally convex shape with the guard upon the interior of the head further away from the handle than the guard towards the exterior. The radius of curvature of the convex shape passes through the end of the handle and the curve extends across the width of the tip. As described above, the guards extend outwards from the blades and the main portion of the head. The curved blades and guards limit the contact of the cutting edges to discrete small areas regulated by how the user turns the handle left or right of the direction of drawing.

An alternate embodiment of the present invention is shown in an isometric view of FIG. 6. The alternate embodiment has an articulating head 5 connected to a curved handle 6. The curved handle offsets the hand of the user above the head 5 so the head can be drawn across a surface in a direction parallel to the handle 6. Generally the blades 4 remain perpendicular to the direction of drawing the handle. The curved handle has a release button 7 that has a stem (not shown) contained within the handle 6 that pushes the head 5 when the button is depressed. The stem ejects the head from the handle as desired by the user, generally before the next patron.

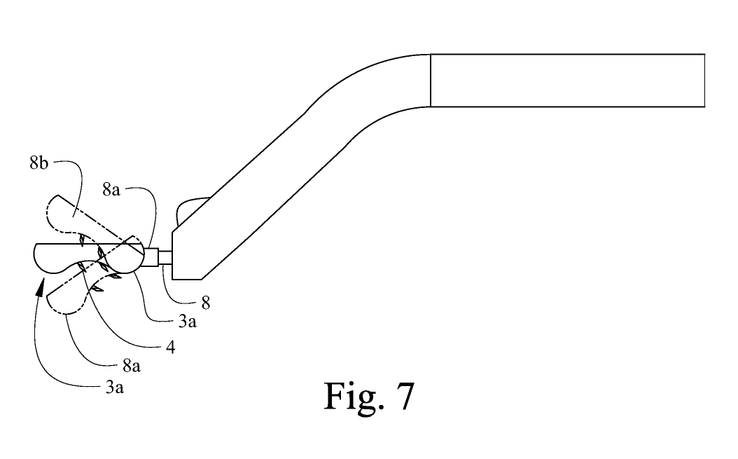

FIG. 7 shows the articulation of the head. The joint 8 allows the head to rotate upwards 8b and downwards 8c in a direction perpendicular to the blades. In the preferred embodiment, the stem connects to the head on a pin 8a that rotates within the head generally on an axis proximate to the longitudinal edge of the head which is forward of the blades 4. Alternatively, the stem connects to a slot that permits rotation of the head without dislodging the stem. As in FIG. 4, the alternate embodiment has a curved handle that includes a release button for detaching the articulated head.

From the aforementioned description, a skin condition remover has been described. The skin condition remover is uniquely capable of removing calluses and other conditions of the skin in a sanitary manner so that successive patrons do not risk contamination from skin cuttings. The skin condition remover and its various components may be manufactured from many materials including but not limited to polymers, high density polyethylene HDPE, polypropylene PP, polyvinyl chloride PVC, nylon, stainless steel, ferrous and non-ferrous metals, their alloys, and composites.

As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

I claim:

1. A device to remove conditions from the skin comprising:
a head, generally rectangular, having at least one blade, said blade having an arcuate thin planar shape and an orientation generally along the longitudinal axis of said head, at least one longitudinal edge, at least one guard locating between said blades and said longitudinal edge; and,
a handle, having a tip and an opposite free end, and connecting to said head upon a longitudinal edge at said tip.

2. The skin condition removal device of claim 1 further comprising:
said blade having a radius of curvature located away from said free end and a cutting edge oriented away from said head and said handle; and,
said guards extending outwards of said head generally more than said blades.

3. The skin condition removal device of claim 2 further comprising:
two of said guards generally spaced apart and parallel upon said head flanking said blades.

4. The skin condition removal device of claim 3 further comprising:
said guards dividing into a plurality of parallel grooves, said grooves passing skin cuttings when said device is in usage and said grooves extending from said blades to the longitudinal edges of said head.

5. The skin condition removal device of claim 3 further comprising:
said guards being continuous thereby directing passage of cuttings during usage of said device.

6. The skin condition removal device of claim 2 further comprising:
said handle attaching substantially across a longitudinal edge of said head behind said blades.

7. The skin condition removal device of claim 2 further comprising:
said handle pivotally attaching to a longitudinal edge of said head.

8. The skin condition removal device of claim 7 wherein said handle attaches ahead of said blades.

9. The skin condition removal device of claim 4 further comprising:
two of said blades, spaced apart and having similar radii of curvature.

10. The skin condition removal device of claim 4 further comprising:
one of said blades;
said blade having a surface generally away from said handle and a plurality of teeth, mutually parallel and spaced apart and generally perpendicular to said surface, and aligned parallel with said guards forming a series of continuous grooves directing cuttings out of said head.
